(19) United States
(12) Patent Application Publication
(10) Pub. No.: US 2019/0234877 A1
(43) Pub. Date: Aug. 1, 2019

(54) METHOD, COMPOSITION AND SENSOR FOR TESTING A SAMPLE FOR THE PRESENCE OF NITRATE OR NITRITE

(71) Applicants: Cambridge Display Technology Limited, Godmanchester (GB); Sumitomo Chemical Company Limited, Tokyo (JP)

(72) Inventor: Jonathan Behrendt, Godmanchester (GB)

(73) Assignees: Cambridge Disaplay Technology Limited, Godmanchester (GB); Sumitomo Chemical Company Limited, Tokyo (JP)

(21) Appl. No.: 16/311,647

(22) PCT Filed: Jun. 9, 2017
(86) PCT No.: PCT/EP2017/064123

§ 371 Date: Dec. 19, 2018

(30) Foreign Application Priority Data
Jun. 23, 2016 (GB) 1610992.8

(51) Int. Cl. G01N 21/64; G01N 21/77; B01L 3/00; C12Q 1/26; G01N 33/18

(52) U.S. Cl. CPC G01N 21/6428; G01N 21/77; B01L 3/502715; G01N 2021/1765; G01N 33/182; G01N 2021/7786; C12Q 1/26

(57) ABSTRACT

A method of testing a sample for the presence of nitrate or nitrite, the method comprising the steps of: forming a mixture by contacting the sample with a composition comprising hydrogen peroxide or a hydrogen peroxide precursor and a fluorescent indicator precursor capable of forming a fluorescent indicator in the presence of peroxynitrite; irradiating the mixture; and measuring fluorescence from the fluorescent indicator. The method may be carried out using a device in which the mixture in a channel or chamber 101 of a microfluidic device is irradiated by light from light source 103 and emission from the fluorescent indicator is detected by photodetector 105.

### RELATED APPLICATIONS

This Application is a national stage filing under 35 U.S.C. § 371 of international PCT application PCT/EP2017/064123, filed Jun. 9, 2017, which claims priority to United Kingdom patent application GB 1610992.8, filed Jun. 23, 2016, each of which is incorporated herein by reference in its entirety.

### FIELD OF THE INVENTION

The present invention relates to a method of detecting nitrate and nitrite by a fluorescent signal, compositions for producing said signal and sensors for carrying out said method.

### BACKGROUND

Nitrate and nitrite ions may be present as a pollutant in the environment, for example nitrate or nitrite originating from agricultural activities, industrial waste or sewage, and these pollutants can present a health risk to humans and animals if ingested. Accordingly, determination of nitrite or nitrate concentration in biological or non-biological samples is important for both environmental and medical reasons.

Lu, C. et al. ‘Flow—injection chemiluminescent determination of nitrite in water based on the formation of peroxynitrite from the reaction of nitrite and hydrogen peroxide’ Anal. Chim. Acta 2002, 474, 107-114 discloses formation of peroxynitrous acid by reaction of nitrite with hydrogen peroxide in an acidic medium and production of weak chemiluminescence upon decomposition to peroxynitrite in basic solution. Chemiluminescence was enhanced with ethyldimethylcetylammonium bromide (EDAB) and uranine.

Lu, C. ‘Chemiluminescent study of carbonate and peroxynitrous acid and its application to the direct determination of nitrite based on solid surface enhancement’ Anal. Chim. Acta 2004, 510, 29-34 discloses a chemiluminescent signal observed when peroxynitrous acid produced by mixing of acidified hydrogen peroxide with nitrite is reacted with carbonate.

Yaqoob, M. et al. ‘Determination of nitrate and nitrite in freshwaters using flow-injection with luminol chemiluminescence detection’ Luminescence 2012, 27, 419-425 discloses a miniaturized system for determination of nitrite and nitrate by microfluidic device with chemiluminescence detection by reaction produced by oxidation of nitrite with hydrogen peroxide in acid medium in the presence of alkaline luminol.

Laitip, N. et al. ‘ Utilization of microfluidic device for determination of nitrate and nitrite in soil samples’ Asian J. Chem. 2013, 25, 6486-6490 discloses reduction of nitrate to nitrite on-line via a copperized cadmium column and then reaction with acidic hydrogen peroxide to form peroxynitrous acid. Chemiluminescence was observed from the oxidation of luminol in an alkaline medium in the presence of the peroxynitrite anion.

Possel, H. et al. ‘2,7-Dihydrodichlorofluorescein diacetate as a fluorescent marker for peroxynitrite formation’ FEBS Lett. 1997, 416, 175-178 discloses oxidation of 2,7-Dihydrodichlorofluorescein by peroxynitrite.

Nussler et al., Nature Protocols 2006, 1, 2223-2226 discloses fluorometric measurement of nitrite/nitrate by 2,3-diaminonaphthalene.

It is an object of the invention to provide a method for detection of nitrate or nitrite suitable for point-of-care testing.

It is a further object of the invention to provide a method for detection of nitrate or nitrite across a wide concentration range.

It is a yet further object of the invention to provide a low cost assay for detection of nitrate or nitrite.

### SUMMARY OF THE INVENTION

The present inventions have found that hydrogen peroxide can be used in an assay for detection of nitrate or nitrite in which the presence of nitrate or nitrite is indicated by a fluorescent indicator.

Accordingly, in a first aspect the invention provides a method of testing a sample for the presence of nitrate or nitrite, the method comprising the steps of:

forming a mixture by contacting the sample with a composition comprising hydrogen peroxide or a hydrogen peroxide precursor and a fluorescent indicator precursor capable of forming a fluorescent indicator in the presence of peroxynitrite;

irradiating the mixture; and

measuring fluorescence from the fluorescent indicator.

The inventors have found that hydrogen peroxide may be formed in situ upon formation of the mixture from a hydrogen peroxide precursor in a solid composition. The hydrogen peroxide precursor comprises two or more, preferably two, reagents for forming hydrogen peroxide.

Accordingly, in a second aspect, the invention provides a solid composition comprising a hydrogen peroxide precursor for forming hydrogen peroxide and a fluorescent indicator precursor capable of forming a fluorescent indicator in the presence of peroxynitrite.

In a third aspect the invention provides a solid formulation comprising a fluorescent indicator precursor capable of forming a fluorescent indicator in the presence of peroxynitrite and a first reagent capable of forming hydrogen peroxide with a second reagent wherein the solid formulation does not comprise the second reagent.

The solid composition or formulation may be provided in or on a device for mixing the sample with the composition and the device may be used as a component of a sensor having a light source and a photodetector.

### DESCRIPTION OF THE DRAWINGS

The invention will now be described in more detail with reference to the figures in which:

FIG. 1 illustrates a sensor according to an embodiment of the invention comprising a light source and a photodetector on opposing sides of a microfluidic device;

FIG. 2 illustrates a sensor according to an embodiment of the invention comprising a light source and a photodetector on the same side of a microfluidic device;

FIG. 3 is a graph of sensor current vs. concentration of nitrite generated from measurements by a method according to an embodiment of the invention for mixtures having different pH values and nitrite concentrations;

FIG. 4 is a graph of sensor current vs. time for a 1 mM sodium nitrite solution generated from measurements according to an embodiment of the invention;

FIG. 5 is a graph of sensor current vs. time for a 10 mM sodium nitrate solution generated from measurements according to an embodiment of the invention in which hydrogen peroxide is present in the composition before contact with the sample;

FIG. 6 is a graph of the rate of increase in sensor current vs. nitrate concentration generated from measurements according to an embodiment of the invention;

FIG. 7A, is a graph of sensor current vs. time for a 10 mM sodium nitrate solution generated from measurements according to an embodiment of the invention in which hydrogen peroxide is generated in situ in the mixture; and

FIG. 7B is a graph of rate of increase of sensor current vs. time for a 10 mM sodium nitrate solution generated from measurements according to an embodiment of the invention in which hydrogen peroxide is generated in situ in the mixture.

### DETAILED DESCRIPTION OF THE INVENTION

The method described herein includes formation of a mixture by bringing a sample into contact with a composition comprising hydrogen peroxide or a hydrogen peroxide precursor comprising two or more reagents for forming hydrogen peroxide and a fluorescent indicator precursor to form a fluorescent indicator, and measuring fluorescence from the fluorescent indicator.

The mixture may be formed by combining the liquid sample and the components of the composition in any order. The liquid sample may be added to the composition to form the mixture. One or more components of the composition may be added to the sample and other components of the composition to form the mixture. At least one component of the mixture is in liquid form. Optionally, one of the composition and the sample is in liquid form, the other being in solid form. Optionally, the sample is in liquid form and the composition is in solid form. If the composition is in solid form then at least one, optionally all, components of the composition are preferably dissolved in the sample to form the mixture.

Optionally, both of the composition and sample are in liquid form.

“Liquid” as described herein means liquid at ambient pressure (1 atmosphere) and ambient temperature (20° C.). A liquid composition or formulation as described herein is preferably a solution or suspension. A liquid sample as described herein includes, without limitation a solution, a colloidal liquid or a suspension. The liquid composition preferably comprises water and one or more components of the composition are preferably water soluble.

The mixture formed by mixing the sample and the composition preferably has a pH of less than 7.0. Optionally, the mixture has a pH of at least 5.0. Optionally, the mixture has a pH in the range of 5.0-6.5 or 5.0-6.0.

If a mixture has, or will have, a pH of 7.0 or more then its pH may be reduced by addition of acid to a liquid sample or by adding the liquid sample to a larger volume of a buffer solution of a lower pH before it is mixed with the composition or after formation of the mixture.

Following mixing of the sample and the composition, the pH of the mixture preferably does not change by more than 0.5, 0.2 or 0.1.

Detection of Nitrite

Detection of nitrite ions in a sample as described herein includes the steps of:

(i) formation of peroxynitrite ions by reaction of nitrite ions with hydrogen peroxide;

(ii) formation of a fluorescent indicator by reaction of a fluorescent indicator precursor with the peroxynitrite ions; and

(iii) measurement of fluorescence from the fluorescent indicator upon irradiation thereof.

The composition may be a solution comprising hydrogen peroxide and the fluorescent indicator precursor that is mixed with the sample to form the mixture. A solution comprising hydrogen peroxide and a separate solution comprising the fluorescent indicator precursor may be mixed with the sample to form the mixture.

In a preferred embodiment, the composition is a solid, optionally a lyophilised solid, comprising or consisting of hydrogen peroxide precursor reagents that react to form hydrogen peroxide in situ in the mixture, and the fluorescent indicator precursor. Optionally, the reagents are an oxidase enzyme and a compound that produces hydrogen peroxide in a reaction catalysed by the oxidase enzyme. The oxidase-catalysed formation of hydrogen peroxide may or may not require the presence of molecular oxygen (02). The reaction preferably occurs in an ambient air environment.

Exemplary reagents for forming hydrogen peroxide are glucose and glucose oxidase; and cholesterol and cholesterol oxidase.

The oxidase may be immobilised on a solid.

In other embodiments, a formulation in solid or liquid form may comprise or consist of the fluorescent indicator precursor and a first reagent for forming hydrogen peroxide wherein the formulation does not comprise a second reagent for forming hydrogen peroxide. The second reagent (and, if required, one or more further reagents) for forming hydrogen peroxide with the first reagent may be added, in solid or liquid form, to the formulation at the time the sample is tested to complete the composition. The second and any further reagents may be combined with the formulation before or after the sample is combined with the formulation.

Maintaining separation of reagents of the hydrogen peroxide precursor until testing may avoid degradation by reaction of the reagents during storage.

The or each reagent for forming hydrogen peroxide may be present in the mixture in a concentration of 1-100 mM, optionally 1-50 mM or 5-50 mM.

Detection of Nitrate

Detection of nitrate ions may be carried out as described with reference to detection of nitrite with the additional, preliminary step of reducing nitrate ions in the sample to nitrite ions that may then react with hydrogen peroxide as described herein.

Nitrate ions in the sample may be reduced to nitrite ions before or after the sample is brought into contact with the composition. The composition or formulation as described herein may comprise a reducing agent for reduction of nitrate ions in the sample to nitrite ions. Optionally, the sample is contacted with the formulation comprising the reducing agent for reduction of nitrate ions in the sample to nitrite ions followed by addition of the reagent or reagents of the composition for formation of hydrogen peroxide which are not present in the formulation.

Exemplary methods for reduction of nitrate ions before the sample is contacted with the composition include photolytic reduction, optionally by UV treatment of the sample; or by use of a cadmium-copper or cadmium mercury couple.

The nitrate reducing agent may be a single material or two or more materials. The nitrate reducing agent preferably selectively reduces nitrate. Any effect of the nitrate reducing agent on peroxynitrite may be accounted for by calibration with a solution of known nitrate concentration and the composition comprising the nitrate reducing agent. Preferably, the nitrate reducing agent is a reducing compound, preferably NADPH, and nitrate reductase which may optionally be used with an electron-transfer material, optionally FAD-Na₂.

The nitrate reducing agent may be provided as a component of a solid or liquid composition as described herein. The or each material of the nitrate-reducing agent may be immobilised on a solid.

Nitrate reduction followed by fluorescent indicator formation as described herein may occur in separate steps in separate reaction vessels or devices, or nitrate reduction may occur in situ upon mixing of the sample with the composition and may occur in or on the same device, optionally in on a lateral flow device or in a microfluidic device.

If the composition or formulation comprises the reducing agent, optionally nitrate reductase and a reducing compound, then the pH of the liquid formed by mixing of the composition or formulation and the sample is preferably in the range of about 5.0-6.5 or 5.0-6.0 for efficient nitrate reduction and fluorescent indicator formation.

The concentration of nitrate reductase in the mixture is preferably at least 1 U/ml, optionally in the range 1-10 or 1-5 U/ml.

Fluorescent Indicator Formation

The peroxynitrite ions formed by reaction of the nitrite ions and the hydrogen peroxide may react with the fluorescent indicator precursor to form the fluorescent indicator.

By “fluorescent indicator” as used herein is meant a material that fluoresces upon irradiation by light.

The presence of the fluorescent indicator may be measured by exciting the indicator with a light source and measuring fluorescence using a photodetector.

The fluorescent indicator may be present in the mixture in a concentration of at least 0.1 mM, optionally 0.1-10 nM. If the method is for detection of nitrate and comprises a step of reduction by a reducing agent, optionally nitrate reductase and NADPH, then the concentration of the fluorescent indicator in the mixture is preferably no more than 1 mM.

The presence of nitrate or nitrite in the sample may be determined from the fluorescence measurement. If nitrate or nitrite is present, its concentration in the sample may be determined.

The fluorescent indicator precursor emits no fluorescence, or comparatively very little fluorescence (preferably less than 10%) compared to the luminance of the fluorescent indicator, upon irradiation with a light source, optionally a light source emitting light within the visible range (390-700 nm) or UV range (greater than 10 nm to less than 390 nm, optionally 100-380 nm).

Preferably, the fluorescent indicator emits light upon irradiation with light in the visible range.

The fluorescent indicator precursor may be, without limitation, selected from the following compounds, each of which may be unsubstituted or substituted with one or more substituents: fluoresceins and salts thereof, rhodamines, coumarins, boron-dipyrromethenes (BODIPYs), naphthalimides, perylenes, benzanthrones, benzoxanthrones; and benzothiooxanthrones.

Exemplary substituents are chlorine, alkyl amino; phenylamino; and hydroxyphenyl. Exemplary fluoresceins include, without limitation, 2,7-dichlorofluorescein, 3′-(p-aminophenyl)fluorescein and 3′-(hydroyphenyl)fluorescein. A fluorescein indicator precursor may react with an oxygen radical to produce a fluorescent, oxidised fluorescein indicator.

The concentration of the fluorescent indicator precursor in the mixture is optionally in the range of 0.01-20 mM, optionally 0.05-20 mM.

The fluorescein may be a compound of formula (Ia) or (Ib) or a salt thereof:

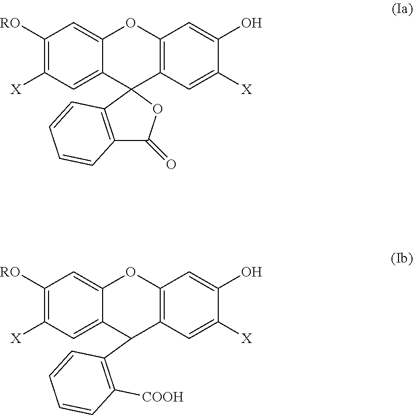

wherein X in each occurrence is independently H, F or Cl and R is H or a substituent, optionally phenyl which may be unsubstituted or substituted with one or more substituents. Substituents of phenyl may be hydroxyl or amino groups.

The fluorescent indicator precursor is preferably soluble in water. The fluorescent indicator precursor is preferably dissolved in the mixture formed upon contact of the sample and the composition.

Sample

The sample described herein may be a biological sample, preferably a biological liquid, optionally blood, urine, saliva, tears, faeces, gastric fluid, bile, sweat, cerebrospinal fluid or amniotic fluid; cell culture media or other biological samples; or non-biological samples for example food, environmental water, e.g. river, sea or rain water, wine, hydroponic solutions, or soil extracts.

Analyte Detection

The sample may be brought into contact with the composition or formulation disposed in or on a device for mixing the liquid sample and the composition.

A liquid composition and liquid sample may be mixed in a microfluidic device.

A solid composition or formulation may be provided in a channel or chamber of a microfluidic device into which a liquid sample may be introduced.

A solid composition or formulation may be immobilised on a surface of a lateral flow device and may be mixed with a liquid sample to form the mixture. The mixture is irradiated with a light source. Any light source may be used including, without limitation, a laser, an inorganic LED or LED array, an arc lamp such as a mercury or xenon arc lamp, a metal halide lamp or one or more organic light-emitting devices (OLEDs). The light source is preferably an OLED.

OLEDs comprise an anode, a cathode and a light-emitting layer comprising an organic light-emitting material between the anode and the cathode. One or more further layers may be provided between the anode and the cathode, optionally one or more charge-transporting, charge injecting or charge-blocking layers. Upon application of a bias between the anode and cathode, light is emitted from the organic light-emitting material. OLEDs may be as described in Organic Light-Emitting Materials and Devices, Editors Zhigang Li and Hong Meng, CRC Press, 2007, the contents of which are incorporated herein by reference.

The fluorescent indicator preferably emits light upon irradiation of light in the visible range of 390-700 nm and the wavelength range of light emitted from the light source may be selected accordingly.

Light emitted from the fluorescent indicator is preferably in the visible range or in the infra-red range (greater than 700 nm, optionally at least 750 nm, up to about 1000 nm) preferably in the visible range.

Light emitted from the fluorescent indicator may be detected by a photodetector, optionally an organic photodetector (OPD), a charge-coupled device (CCD) or a photomultiplier, preferably an OPD or CCD.

An OPD comprises an anode, a cathode and an organic semiconducting region between the anode and cathode. The organic semiconducting region may comprise adjacent electron-donating and electron-accepting layers or may comprise a single layer comprising a mixture of an electron-accepting material and an electron-donating material. One or more further layers may be provided between the anode and the cathode. Conversion of light incident into electrical current may be detected in zero bias (photovoltaic) mode or reverse bias mode. OPDs may be as described in Ruth Shinar & Joseph Shinar “Organic Electronics in Sensors and Biotechnology” McGraw-Hill 2009, the contents of which are incorporated herein by reference.

The mixture may be irradiated continuously or at multiple points in time for detection of fluorescence from the fluorescent indicator.

Measurements of fluorescence may be made at multiple points in time. Such multiple measurements may be used for a kinetic assay. Optionally, readings of a kinetic assay are taken over a period of up to 30 minutes, 20 minutes or 10 minutes.

A single measurement may be made after a predetermined period of time (an end-point assay). Optionally, the end-point assay reading is taken after a period of up to 30 minutes, 20 minutes or 10 minutes.

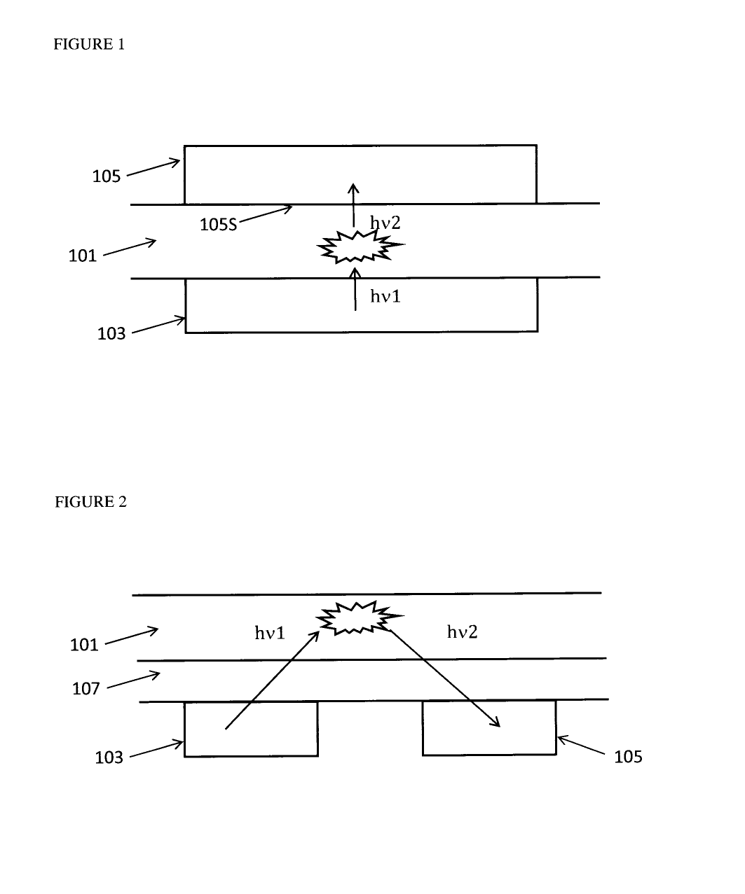

FIG. 1, which is not drawn to any scale, illustrates a sensor suitable for use in a method as described herein comprising a light source, a photodetector and a microfluidic device.

In use, a sample is contacted with the composition described herein in channel or chamber 101 of a microfluidic device and is illuminated with light from light source 103 of wavelength hν1. If the fluorescent indicator has been formed then the light from the light source is absorbed and re-emitted by the fluorescent indicator as light of longer wavelength hν2 which may be detected by photodetector 105 having a surface 105S on which light is incident.

In the embodiment of FIG. 1, the light source 103 is provided on a first surface of the microfluidic device and the photodetector 105 is provided on an opposing, second surface.

A filter (not shown) may be provided between the light source and the photodetector to eliminate some or all wavelengths of light other than a wavelength range emitted by the fluorescent indicator.

A filter (not shown) may be provided between the light source and the mixture to eliminate some or all wavelengths of light other than a wavelength range absorbed by the fluorescent indicator.

FIG. 2, which is not drawn to any scale, illustrates another sensor other arrangement in which the light source 103 and photodetector 105 are provided on a first surface of the microfluidic device. In this embodiment, light emitted from the light source may be prevented from reaching the photodetector 105 by use of a highly absorbing (black) layer on or over a second surface of the microfluidic device opposing the first surface and/or by use of a filter on or over the surface of the photodetector on which light is incident.

The light source 103 and photodetector 105 are provided on a common substrate 107, such as a glass or plastic substrate, provided adjacent to the first surface of the microfluidic device. In another embodiment, the first surface of a microfluidic device may form a common substrate on which the light source and photodetector are formed. In a yet further embodiment, light source 103 and photodetector 105 may be provided on separate substrates on the first surface.

In the case where the light source is an OLED and the photodetector is an OPD, the OLED and photodetector may be formed on a common substrate which is then brought adjacent to the first surface of the microfluidic device to form the sensor. The OPD and OLED of this embodiment may be formed using a common transparent anode layer on the substrate, optionally a common indium tin oxide layer.

It will be appreciated that the light source and photodetector may be provided in a wide range of arrangements to sense emission of fluorescent light from the fluorescent indicator and may be used with, without limitation, filters, light-absorbing layers, light-reflecting layers, lenses, optical fibres and combinations thereof.

The sensor may have a modular structure in which the microfluidic device is separable from the light source and/or photodetector. Optionally, the microfluidic device of the sensor comprises a single use glass or transparent plastic microfluidic chip which may be removed and replaced with another chip.

Optionally, the microfluidic device is not modular, the entire sensor being a single-use sensor.

The or each component of the composition or formulation may be introduced into a microfluidic device from a solution or suspension comprising the component dissolved or suspended therein and then removing the solvent or solvents of the solution or suspension, optionally by lyophilising the solution or suspension.

The or each component of the composition or formulation may be absorbed onto or into a lateral flow device by applying the components of the composition or formulation from one or more solutions or suspensions onto a surface of the device followed by evaporation of the solvent or solvents of the solution or suspension.

The sensor may be a portable device. The sensor may be a handheld device.

FIGS. 1 and 2 illustrate a sensor comprising a microfluidic device in which the sample is brought into contact with the composition, however it will be appreciated that other apparatus may be used for mixing the liquid sample with the composition, for example a lateral flow device having a surface on which the composition or formulation is immobilised in solid form.

In the case where a device comprises a formulation in solid form, the reagent or reagents for forming hydrogen peroxide not present in the formulation may be added before or after the sample is contacted with the formulation, or added with the sample. A kit may be provided, the kit comprising a device comprising the formulation in solid form and the reagent or reagents for forming hydrogen peroxide not present in the formulation.

FIGS. 1 and 2 illustrate a sensor having only one light source and only one photodetector. There may be more than one light source for each detector.

The sensor may comprise one channel for detection of nitrate and another channel for detection of nitrate.

The sensor may be a multi-channel microfluidic device wherein at least one channel is configured to detect nitrate and/or nitrite ions as described herein, the sensor comprising one or more further channels each being configured to detect an analyte other than nitrate or nitrite ions.

The sensors described herein may enable detection of nitrate and/or nitrite at low concentration and/or across a wide analyte concentration range. The nitrate or nitrite concentration in the sample for analysis may be in the range of about 0.1-10 mM.

The compositions described herein may be used in an assay for detection of nitrate and/or nitrite ions in point-of-care sensors.

### EXAMPLES

All reagents were purchased from Sigma Aldrich.

### Example 1: Formation of 2,7-Dichlorofluorescein Fluorescent Indicator Precursor

2,7-Dichlorofluorescein diacetate was dissolved in DMSO at a concentration of 1 mg/mL (2 mM). To 50 μL of this solution was added methanol (50 μL) and 2M aqueous potassium hydroxide (50 μL) and the mixture was left to stand at room temperature for 1 hour (final concentration of detection reagent is 0.67 mM).

### Example 2

Sodium nitrite and 2,7-dichlorofluorescein were mixed with sodium acetate buffer (0.1 M, pH 5.5), followed by addition of 10 μL of hydrogen peroxide solution (1058 mM, aqueous) to 990 μL of this solution. Assay solutions thus formed had nitrite concentrations of 0.1, 1 and 10 mM and a 2,7-dichlorofluorescein concentration of 0.1 mM. After mixing, ˜130 μL of the solution was used to entirely fill a microfluidic flow cell (20×9 mm area with an optical pathlength of 0.5 mm). This flow cell was placed within the OLED/OPD detection apparatus shown in FIG. 1 having a short pass filter between the OLED and the microfluidic flow cell and a long pass filter between the microfluidic flow cell and the OPD and fluorescence was measured after the specified reaction times (t=0 min being the time of adding hydrogen peroxide to the assay) using a drive current of 20 mA, an OPD bias of 0 V and a pulse time of 100 ms. The peak emission wavelength of the OLED used as the excitation source is 480 nm.

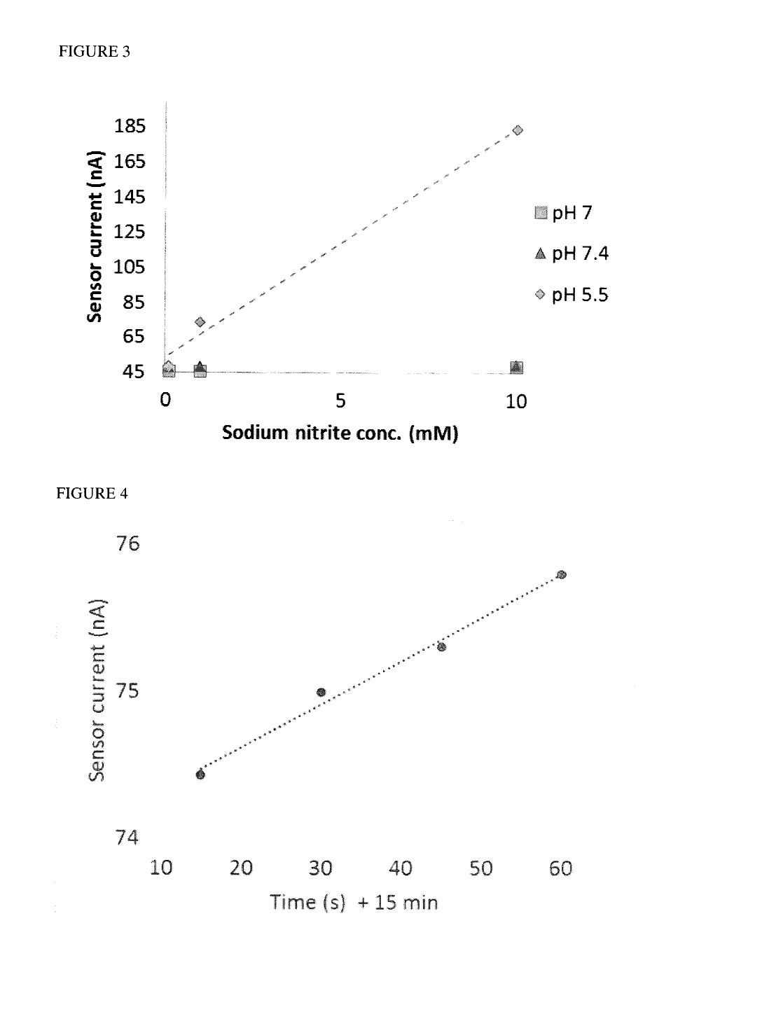

With reference to FIG. 3, where fluorescence of the assay solutions was measured 15 minutes after addition of hydrogen peroxide, there is a linear relationship between sensor current (corresponding to intensity of fluorescence from the fluorescent indicator) and concentration of nitrite.

With reference to FIG. 4, the sensor current increases linearly with time.

The same experiment was carried out using 0.1 phosphate buffer with pH of 7.0 or 7.4 instead of pH 5.5 sodium acetate buffer, however no increase in fluorescence from the fluorescent indicator was detected at these pH values.

The OLED was supported on a glass substrate and comprised a transparent anode, a hole injection layer, a polymeric hole-transporting layer, a light-emitting layer comprising a fluorescent blue light-emitting polymer and a cathode. The peak emission wavelength of the OLED was 480 nm.

The OPD was supported on a glass substrate and comprised a transparent anode, a hole transporting layer, a layer of a mixture of a donor polymer illustrated below and a C70 fullerene acceptor material and a cathode.

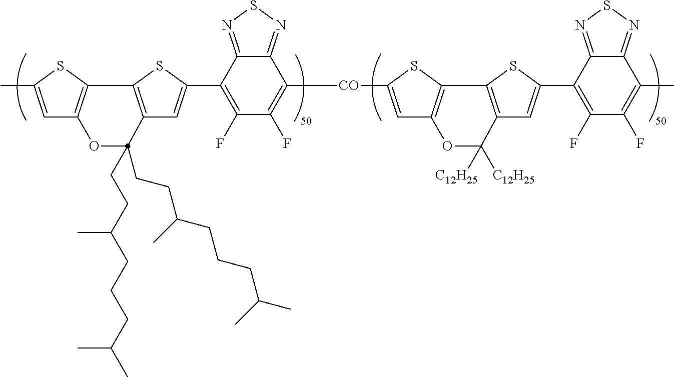

### Example 3

Nitrate reductase, NADPH, hydrogen peroxide, FAD-Na₂, sodium nitrate and 2,7-dichlorofluorescin were mixed with sodium acetate buffer (0.1 M, pH 5.5) to form aqueous solution having a pH of 5.5 To 990 μL of this solution was added 10 μL of hydrogen peroxide solution (1058 μM, aqueous). The assay solution thus formed contained 0.3 U/mL of nitrate reductase, 1 mM of NADPH, 0.1 mM of FAD-Na₂, 0.1 mM dichlorofluorescein and 10 mM of potassium nitrate. After mixing, the fluorescence of the solutions was measured as described in Example 2.

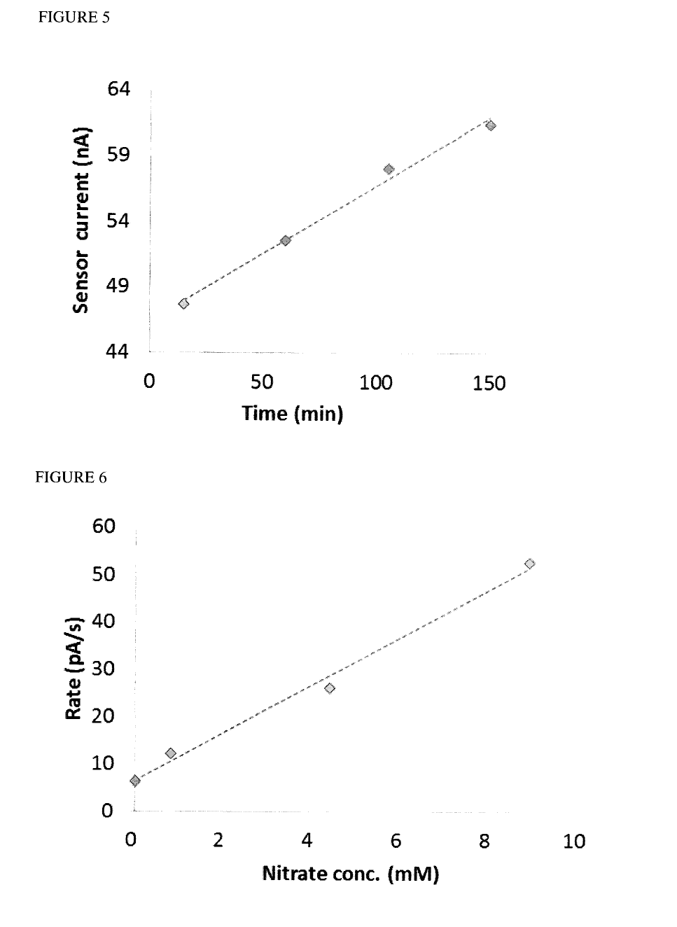

With reference to FIG. 5, the sensor current increases linearly with time.

### Example 4

Solutions were formed as described in Example 3 in which concentrations of NADPH, nitrate reductase and the fluorescent indicator precursor were varied. After mixing, the fluorescence of solutions was measured as described in Example 2.

With reference to Table 1, the rate at which the sensor current increases (pA/s) may be controlled by selecting the concentration of the components of the mixture. A relatively high concentration of nitrate reductase and/or a relatively low concentration of the fluorescent indicator precursor gives a relatively raid increase in sensor current.

TABLE 1

| | Fluorescent | | |
|---|---|---|---|
| | indicator | Nitrate reductase | Rate of signal |
| NADPH (mM) | precursor (mM) | (U/mL) | increase (pA/s) |
| 10 | 1 | 0.3 | 11 |
| 1 | 1 | 0.3 | 4.1 |
| 10 | 10 | 0.3 | 9.9 |
| 10 | 1 | 3 | 25 |
| 10 | 0.1 | 3 | 53 |

### Example 5

Solutions were formed as described in Example 3 in which the concentration of sodium nitrate was varied between 0.1-10 mM. 5 minutes after addition of hydrogen peroxide, the rate of sensor current increase was measured over a period of 5 minutes (i.e. from the period of 5-10 minutes following start of the assay).

Measurement was carried out as described in Example 2.

With reference to FIG. 6, the rate of increase in sensor current is proportional across the measured nitrate concentration range of about 0.1-10 mM.

### Example 6

Solutions were formed as described in Example 3 except that hydrogen peroxide was replaced with glucose and glucose oxidase for in situ formation of hydrogen peroxide.

The solutions contained glucose in a concentration of 10, 25 or 250 mM.

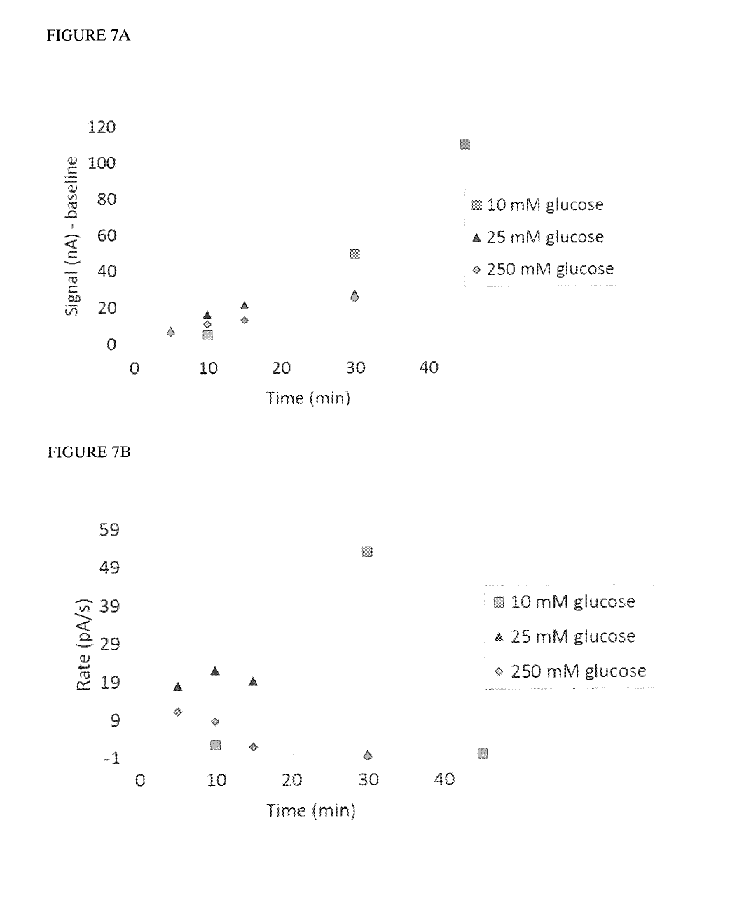

With reference to FIG. 7A, a signal is detected most quickly at higher glucose concentrations, although the strongest signal is ultimately generated at lower glucose concentration. With reference to FIG. 7B, the rate at which the signal increases is initially higher at high glucose concentration but is ultimately highest at lower glucose concentration.

Without wishing to be bound by any theory, nitrate reductase may degrade over time at high hydrogen peroxide concentrations.

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. A method of testing a sample for the presence of nitrate or nitrite, the method comprising the steps of:
forming a mixture by contacting the sample with a composition comprising hydrogen peroxide or a hydrogen peroxide precursor and a fluorescent indicator precursor capable of forming a fluorescent indicator in the presence of peroxynitrite;
irradiating the mixture; and
measuring fluorescence from the fluorescent indicator.

2. A method according to claim 1 wherein the composition comprises a hydrogen peroxide precursor.

3. A method according to claim 2 wherein the hydrogen peroxide precursor comprises an oxidase and a compound capable of forming hydrogen peroxide by action of the oxidase on the compound.

4. A method according to claim 3 wherein the compound is glucose and the oxidase is glucose oxidase.

5. A method according to claim 1 wherein the method is for testing the sample for the presence of nitrate, the method comprising reduction of the nitrate ions to nitrite ions.

6. A method according to claim 5 wherein the nitrate ions are reduced to nitrite ions before formation of the mixture.

7. A method according to claim 5 wherein the composition comprises a reducing agent for reducing the nitrate ions to nitrite ions.

8. A method according to claim 7 wherein the reducing agent comprises nitrate reductase and NADPH.

9. A method according to claim 1 wherein the fluorescent indicator precursor is a fluorescein.

10. A method according to claim 1 wherein the mixture is formed by bringing the sample in liquid form into contact with the composition in solid form.

11. A method according to claim 10 wherein the composition is in lyophilised form.

12. A method according to claim 1 wherein the hydrogen peroxide precursor comprises a first reagent and a second reagent for forming hydrogen peroxide, wherein the mixture is formed by contacting the sample in liquid form and the second reagent with a solid formulation which comprises the fluorescent indicator precursor and the first reagent and which does not comprise the second reagent.

13. A method according to claim 12 wherein the formulation is in lyophilised form.

14. A method according to claim 1 wherein the sample is a liquid sample which is brought into contact with the composition in a microfluidic device or lateral flow device.

15. A method according to claim 10 wherein the solid composition is provided in the microfluidic device or lateral flow device.

16. A method according to claim 12 wherein the solid formulation is provided in the microfluidic device or lateral flow device.

17. A method according to claim 1 wherein the sample is irradiated with visible light.

18. A method according to claim 1 wherein a concentration of the analyte is determined from the fluorescence measurement.

19. A method according to claim 1 wherein the sample and composition are brought into contact in a sensor comprising a device for mixing the liquid sample and the composition; a light source for irradiation of the mixture; and a photodetector for detection of light emitted by the fluorescent indicator.

20. A method according to claim 19 wherein the device is a microfluidic device or lateral flow device.

21-27. (canceled)
